(19) United States
(12) Patent Application Publication
(10) Pub. No.: US 2013/0157253 A1
(43) Pub. Date: Jun. 20, 2013

(54) DETECTION OF CYTOMEGALOVIRUS DNA USING AMPLIFICATION FROM BLOOD SAMPLES

(71) Applicant: Mei Wang Baker, Madison, WI (US)

(72) Inventor: Mei Wang Baker, Madison, WI (US)

(21) Appl. No.: 13/327,977

(22) Filed: Dec. 16, 2011

(51) Int. Cl. C12Q 1/70

(52) U.S. Cl. 435/5

(57) ABSTRACT

Described are methods and kits for detecting cytomegalovirus DNA in liquid and dried blood samples. Primer and probe combinations for CMV detection are described as well as methods for isolating DNA from blood samples.

### FIELD OF THE DISCLOSURE

The present disclosure is related to compositions and methods for the detection of cytomegalovirus DNA.

### BACKGROUND

Cytomegalovirus, CMV, is a common virus that typically results in infections with no outward symptoms. However, the virus can be deadly in people with compromised immune systems or in babies congenitally infected. The CDC reports that about 1 in 150 children is born with congenital CMV infections, and about 1 in every 5 children born with the virus will suffer permanent physical problems; such as hearing loss or developmental disabilities. Presently, newborn blood screening can be done to determine the presence of viral DNA in blood, urine, or saliva. However, the sensitivity of the tests is not optimal; the test takes 2-3 days to run; and because of the low levels of virus in blood and the small amount of blood present in the dried blood spot, diagnosing CMV infection in dried blood samples is not routinely done. Babies who test positive for CMV infection need to have their hearing and vision checked regularly. The antiviral drug, ganciclovir, can be prescribed to infants who suffer severe symptoms of congenital CMV infections. This drug has very severe side effects, so it is used as a treatment of last resort.

Screening for disease is a paradigm of modern medical practice. In particular, newborn or neonatal screening is the practice of testing newborns for certain harmful or potentially fatal disorders that are not otherwise apparent at birth. Generally, blood drops are obtained from the heel or the ear and then absorbed onto filter paper to produce a dried blood spot collection card, often referred to as a Guthrie card. The dried blood spots are tested for a variety of individual diseases and conditions, including those of metabolic, genetic, and/or hormonal origin. Because most of the United States mandate newborn screening, there is significant motivation, from both the economic and medical practice perspectives, for the development of rapid screening methods having a low overall cost. This testing saves millions of dollars per year in health care costs for treating people who would suffer the long-term effects of these disorders and diseases if not treated upon birth.

What is needed are improved methods and kits for the detection of CMV infections, particularly the screening of newborns for CMV infections.

### BRIEF SUMMARY

In one aspect, a method of screening for CMV in DNA isolated from a blood sample comprises amplifying a sequence of CMV DNA in the DNA isolated from the blood sample by contacting the DNA isolated from the blood sample with primers that are 15 to 30 nucleotides in length and comprise SEQ ID NO: 1 and SEQ ID NO: 2 under amplification conditions to produce an amplification product, and detecting the presence of the amplification product as an indication of the presence of CMV in the blood sample.

In yet another aspect, a kit for the detection of CMV a blood sample comprises a DNA elution buffer, nucleic acid amplification reagents, a primer pair for amplification of CMV, wherein the primer pair are 15 to 30 nucleotides in length and comprise SEQ ID NO: 1 and SEQ ID NO: 2, and instructions for use.

### BRIEF DESCRIPTION OF THE DRAWINGS

FIG. 1 is a conventional PCR reaction done on DNA extracts from CMV spiked dried filter paper to determine the detection limits of the methods disclosed herein.

FIG. 2 shows the results of a real-time PCR experiment done on filter paper spiked with 6.5-650 copies of CMV DNA.

FIG. 3 shows the results of a real-time PCR experiment done on dried blood spots containing CMV DNA with low (2-6 copies) and high (38-119) concentration.

The above-described and other features will be appreciated and understood by those skilled in the art from the following detailed description, drawings, and appended claims.

### DETAILED DESCRIPTION

Described herein is a sensitive CMV detection assay that can be used, for example, with dried blood newborn screening specimens with a detection capability of less than 10 CMV DNA copies per sample. Because CMV is not genomic DNA and is present at a low copy number, detection of CMV in blood samples, particularly in 3.2 mm dried blood samples, has been extremely challenging. Using previous methods, 2-3 dried blood samples were necessary for CMV detection, which is unacceptable in routine newborn screening. In one embodiment, the DNA detection is performed without washing the extracted DNA, which aids in retaining the low copy number CMV DNA in the sample. In another embodiment, the method is performed with a one-step DNA extraction buffer which allows for fast and reliable diagnosis of CMV infections, particularly in dried blood spots. In yet another embodiment, the CMV assay is performed simultaneously with detection of SCID.

In one aspect, a method of screening for CMV in DNA isolated from a blood sample comprises amplifying a sequence of CMV DNA in the DNA isolated from the blood sample by contacting the DNA isolated from the blood sample with a forward and a reverse primer under amplification conditions to produce an amplification product, and detecting the presence of the amplification product as an indication of the presence of CMV in the blood sample.

As used herein, a primer and a probe are short polynucleotides, generally having a length of 15 to 30 nucleotides, specifically 15 to 25 nucleotides, more specifically, 15 to 20 nucleotides and most specifically 17 or 18 nucleotides. Several primers and probes were selected for analysis, however the primers and probes disclosed herein provided superior results in the amplification of CMV DNA. The primer sequences disclosed herein can be lengthened by adding additional sequences from the CMV genome (NC_{—}006273.2).

In a specific embodiment, the forward and reverse primers for the detection of CMV comprise

| | | |
|---|---|---|
| Forward | 5′-GCCGGCGGTATCG-3′ | SEQ ID NO: 1 |
| Reverse | 5′-GAGCCCGACTTTAC-3′. | SEQ ID NO: 2 |

In one embodiment, the primers for the amplification of CMV are 15 to 30 nucleotides in length and comprise SEQ ID NO: 1 and SEQ ID NO: 2. In another embodiment, the primers for the amplification of CMV are 15 to 25 nucleotides in length and comprise SEQ ID NO: 1 and SEQ ID NO: 2. In yet another embodiment, the primers for the amplification of CMV are 15 to 20 nucleotides in length and comprise SEQ ID NO: 1 and SEQ ID NO: 2.

In a specific embodiment, the forward and reverse primers for the detection of CMV comprise

| | | |
|---|---|---|
| Forward | 5′-CAGCCGGCGGTATCGAT-3′ | SEQ ID NO: 3 |
| Reverse | 5′-CCGAGCCCGACTTTACCA-3′. | SEQ ID NO: 4 |

In one embodiment, amplifying is a real-time PCR amplification including a probe as follows:

| | | |
|---|---|---|
| Probe | 5′-CTTGTTGCGGTACT-3′ | SEQ ID NO: 5 |

In one embodiment, the probe for the amplification of CMV is 15 to 30 nucleotides in length and comprises SEQ ID NO: 5. In another embodiment, the probe for the amplification of CMV is 15 to 25 nucleotides in length and comprises SEQ ID NO: 5. In yet another embodiment, the probe for the amplification of CMV is 15 to 20 nucleotides in length and comprises SEQ ID NO: 5.

In a specific embodiment, the probe for amplification of CMV is:

| | | |
|---|---|---|
| Probe | 5′-ATCTTGTTGCGGTACTGG-3′ | SEQ ID NO: 6 |

In one embodiment, the probe of SEQ ID NOs: 4-6 contains a detectable label. In one embodiment, the probe contains a reporter dye covalently linked at the 5′ terminus and a nonfluorescent quencher covalently linked at the 3′ terminus. The 5′ reporter can be a FAMT^{M} or VIC® reporter dye and the 3′ nonfluorescent quencher can be MGB, a minor groove binding moiety.

As used herein, the term “amplification conditions” refers to those conditions that promote annealing and extension of one or more targeted polynucleotide sequences. In addition to suitable buffers and an enzyme that has polymerase activity, annealing conditions require the addition of nucleotides for amplification. It is well known in the art that such annealing is dependent in a rather predictable manner on several parameters, including temperature, ionic strength, sequence length, complementarity, and G:C content of the primers.

In one embodiment, the test to detect CMV can include simultaneous detection of SCID. The inventor of the present disclosure developed a genetic test that can identify those suffering from SCID, severe combined immunodeficiency disease or “bubble boy disease.” This disease is characterized by defects in the T and B cell lymphocyte systems, leaving the person suffering from the disease highly susceptible to infections. These infections can escalate into life threatening conditions. If detected early, doctors understand what they are treating and parents can be aware of the infection risks. The most difficult steps in developing the test have involved reducing signal to noise problems. The DNA biomarker for SCID is called a T-cell receptor excision circle (TREC) generated by the DNA recombination process. TRECs do not replicate because they are episomal DNA circles. So TRECs are diluted out as cells undergo many divisions. It is imperative that enough DNA is eluted from the dried blood spot to ensure detection of low copy numbers of this DNA marker and that the eluted DNA has sufficient purity for further processing and analysis steps.

The forward and reverse primers for the detection of SCID are as follows

| SEQ ID NO: 7 | |
|---|---|
| Forward | 5′- ACACCTCTGGTTTTTGTAAA -3′ |
| SEQ ID NO: 8 | |
| Reverse | 5′- CAGGTGCCTATGCAT -3′ |

In one embodiment, the primers for the amplification of SCID are 15 to 30 nucleotides in length and comprise SEQ ID NO: 7 and SEQ ID NO: 8. In another embodiment, the primers for the amplification of SCID are 15 to 25 nucleotides in length and comprise SEQ ID NO: 7 and SEQ ID NO: 8. In yet another embodiment, the primers for the amplification of SCID are 15 to 20 nucleotides in length and comprise SEQ ID NO: 7 and SEQ ID NO: 8.

Specific forward and reverse primers for the detection of SCID are as follows

| SEQ ID NO: 9 | |
|---|---|
| Forward | 5′- TGACACCTCTGGTTTTTGTAAAGG -3′ |
| SEQ ID NO: 10 | |
| Reverse | 5′- TGCAGGTGCCTATGCATCA -3′ |

In one embodiment, amplifying of SCID is a real-time PCR amplification including a probe as follows:

| SEQ ID NO: 11 | |
|---|---|
| Probe | 5′-CACTCCTGTGCA-3′ |

In one embodiment, the probe for the amplification of SCID is 15 to 30 nucleotides in length and comprises SEQ ID NO: 11. In another embodiment, the probe for the amplification of SCID is 15 to 25 nucleotides in length and comprises SEQ ID NO: 11. In yet another embodiment, the probe for the amplification of SCID is 15 to 20 nucleotides in length and comprises SEQ ID NO: 11.

In one embodiment, the probe for amplification of SCID is:

| | | |
|---|---|---|
| Probe | 5′- CCCACTCCTGTGCACG -3′ | SEQ ID NO: 12 |

In one embodiment, the amplification also contains control primers/probes for the detection of β-actin. For example,

| SEQ ID NO: 13 | |
|---|---|
| Forward | 5′- ATTTCCCTCTCAGGAATGGA -3′ |
| SEQ ID NO: 14 | |
| Reverse | 5′- CGTCACACTTCATGATGGAGTTG -3′ |

In one embodiment, amplifying of β-actin is a real-time PCR amplification including a probes as follows:

| SEQ ID NO: 15 | |
|---|---|
| Probe | 5′- GTGGCATCCACGAAACTA -3′ |

The CMV primers and probes described herein are suitable for detection of CMV DNA in samples such as liquid and dried blood samples, however, when washing steps are eliminated, particularly good results are obtained. In one embodiment, DNA is eluted with a one-step elution buffer as described herein. The elution method described herein eliminates the numerous wash steps prior to and subsequent to eluting the DNA from the sample into solution that are included in prior art protocols. Unexpectedly, eliminating the wash steps has no detrimental effect on the signal-to-noise ratio and the DNA eluted in the one-step process can be used directly in subsequent enzymatic DNA amplification steps with no further purification. In addition, the optional agitation step aids to release the DNA from blood samples. The simplicity of the process described herein advantageously lends itself more readily to automation than prior art processes.

In one embodiment, a method of eluting DNA from a blood sample comprises adding a one-step elution buffer to the blood sample to form a mixture, and heating the mixture for a time sufficient to elute the DNA from the blood sample to form an eluted DNA solution. Optionally, the eluted DNA solution is cooled at a temperature of at least about 4° C. for at least 5 minutes. In one embodiment, a method of eluting DNA from a blood sample consists essentially of adding a one-step elution buffer to the blood sample to form a mixture, and heating the mixture for a time sufficient to elute the DNA from the blood sample to form an eluted DNA solution. Optionally, the eluted DNA solution is cooled at a temperature of at least about 4° C. for at least 5 minutes. As used herein, the term mixture includes solutions of liquid blood samples in the elution buffer as well as a dried blood sample suspended in the elution buffer. In another embodiment, a method of eluting DNA from a blood sample consists of adding a one-step elution buffer to the blood sample to form a mixture, and heating the mixture for a time sufficient to elute the DNA from the blood sample to form an eluted DNA solution. Heating is performed at a temperature of 90° C. to 99° C., specifically 94° C. to 99° C., and more specifically at 95° C. The time sufficient to elute the DNA is generally about 10 to about 40 minutes. The eluted sufficient to elute the DNA from the blood sample to form an eluted DNA solution, and analyzing the eluted DNA solution without further processing after elution such as washing or DNA solution is suitable for direct use in an enzymatic DNA amplification reaction. Heating is optionally performed with agitation. Optionally, the eluted DNA solution is cooled at a temperature of at least about 4° C. for at least 5 minutes. Optionally, the eluted DNA solution is centrifuged to remove particulates such as debris from dried blood filter paper.

In another embodiment, a method of analyzing DNA from a blood sample comprises adding a one-step elution buffer to the blood sample to form a mixture, heating the mixture for a time without further processing after elution such as washing or the use of an additional buffer. In another embodiment, a method of analyzing DNA from a blood sample consists essentially of adding a one-step elution buffer to the blood sample to form a mixture, heating the mixture for a time without further processing after elution such as washing or the use of an additional buffer. In yet another embodiment, a method of analyzing DNA from a blood sample consists of adding a one-step elution buffer to the blood sample to form a mixture, heating the mixture for a time sufficient to elute the DNA from the blood sample to form an eluted DNA solution, and analyzing the eluted DNA solution without further processing after elution such as washing or the use of an additional buffer. Heating is optionally performed with agitation. Optionally, the eluted DNA solution is cooled at a temperature of at least about 4° C. for at least 5 minutes. Optionally, the eluted DNA solution is centrifuged to remove particulates such as debris from dried blood filter paper.

As used herein, the term consisting essentially of is meant to eliminate the use of additional washing steps and/or buffers during elution and prior to analysis. The advantage of this method is that with the addition of a one-step elution buffer and heating, a sufficient quality and quantity of DNA is produced for further analysis, such as, for example, a PCR reaction. Also, unexpectedly, the eluted DNA solution which contains DNA as well as the one-step elution buffer does not interfere with the enzymes used for DNA amplification when added to an amplification reaction. After elution of the DNA, the eluted DNA solution is optionally centrifuged to remove debris from dried blood filter paper and any particulate matter.

In another embodiment, a method of analyzing DNA from a plurality of blood samples comprises disposing the each of the plurality of blood samples into a different well of a first multi-well plate, adding a one-step elution buffer to each well of the first multi-well plate to form a mixture in each well of the multi-well plate, heating the mixture for a time sufficient to elute the DNA from the plurality of blood samples to form an eluted DNA solution in each well of the multi-well plate, and transferring each eluted DNA solution to a well of a second multi-well plate without washing, wherein each well of the second multi-well plate contains a reaction mixture and primers for an enzymatic DNA amplification reaction, and performing an enzymatic DNA amplification reaction in each well of the second multi-well plate that contains an eluted DNA solution.

In another embodiment, a method of analyzing DNA from a plurality of blood samples consists essentially of disposing the each of the plurality of blood samples into a different well of a first multi-well plate, adding a one-step elution buffer to each well of the first multi-well plate to form a mixture in each well of the multi-well plate, heating the mixture for a time sufficient to elute the DNA from the plurality of blood samples to form an eluted DNA solution in each well of the multi-well plate, and transferring each eluted DNA solution to a well of a second multi-well plate without washing, wherein each well of the second multi-well plate contains a reaction mixture and primers for an enzymatic DNA amplification reaction, and performing an enzymatic DNA amplification reaction in each well of the second multi-well plate that contains an eluted DNA solution. Optionally, the eluted DNA solution is cooled at a temperature of at least about 4° C. for at least 5 minutes. In another embodiment, the method is performed in a single multi-well plate and the reaction mixture and primers for the enzymatic DNA amplification reaction are added to the eluted DNA solution in each well of the multi-well plate.

In one embodiment, the one-step elution buffer is Gentra Elution Solution (Qiagen®), which has a pH of about 13. In another embodiment, the one-step elution buffer consists essentially of 5 to 22.5 mM potassium in the form of KOH, KCl, or a combination thereof, and 7.5 to 30 mM of a base having a buffering range of 7.0 to 9.5, wherein the pH of the one-step elution buffer is about 9 to about 13, specifically 10 to 13, and more specifically 11 to 12. In another embodiment, the one-step elution buffer consists of 5 to 22.5 mM potassium in the form of KOH, KCl, or a combination thereof, and 7.5 to 30 mM of a base having a buffering range of 7.0 to 9.5, wherein the pH of the one-step elution buffer is about 9 to about 13, specifically 10 to 13, and more specifically 11 to 12. In one embodiment, the base is Tris base, which has a buffering range of 7.0 to 9.2. Additional suitable buffers that buffer in a pH range of 7.0 to 9.5 include DIPSO (3-[N,N-bis(2-Hydroxyethyl)amino]-2-hydroxypropanesulfonic Acid Sodium Salt) (7.0-8.2), MOBS (3-(N-morpholino)propanesulfonic acid) (6.9-8.3), TAPSO (3-[N-Tris(hydroxymethyl)methylamino]-2-hydroxypropanesulfonic Acid) (7.0-8.2), HEPPSO (4-(2-Hydroxyethyl)piperazine-1-(2-hydroxypropanesulfonic acid) (7.1-8.5), POPSO (piperazine-1,4-bis(2-hydroxypropanesulfonic acid)dihydrate) (7.2-8.5), TEA (triethanolamine) (7.3-8.3), EPPS (4-(2-hydroxyethyl)piperazine-1-propanesulfonic acid) (7.3-8.7), TRICINE (N-[Tris(hydroxymethyl)methyl]glycine, 3-[(3-Cholamidopropyl)dimethylammonio]propanesulfonic acid) (7.4-8.8), Glycylglycine (7.5-8.9), BICINE (N,N-Bis(2-hydroxyethyl)glycine) (7.6-9.0), HEPBS (N-(2-Hydroxyethyl)piperazine-N′(4-butanesulfonic acid) (7.6-9.0), TAPS(N-Tris(hydroxymethyl)methyl-3-aminopropanesulfonic Acid) (7.7-9.1), AMPD (2-Amino-2-methyl-1,3-propandiol) (7.8-9.7), TABS (N-tris[hydroxymethyl]methyl-4-aminobutanesulfonic acid) (8.2-9.6), AMPSO(N-(1,1-dimethyl-2-hydroxyethyl)-3-amino-2-hydroxy-propanesulfonic acid) (8.3-9.7), CHES (N-Cyclohexyl-2-aminoethanesulfonic acid) (8.6-10.0), and combinations thereof.

In another embodiment, the one-step elution buffer consists essentially of 2.5 to 10 mM KOH, 7.5 to 30 mM Tris base and 2.5 to 12.5 mM KCl. In another embodiment, the one-step elution buffer consists of 2.5 to 10 mM KOH, 7.5 to 30 mM Tris base and 2.5 to 12.5 mM KCl. In one embodiment, the one-step elution buffer consists of 5 mM KOH, 15 mM Tris base and 10 mM KCl and has a pH of 11 to 12.

The DNA elution methods described herein are particularly suitable for the elution of DNA from blood samples. The blood sample is either a liquid blood sample or a dried blood sample. In one embodiment, a dried blood sample is a dried blood spot specimen on a filter paper card, also called a Guthrie card. In one embodiment, the sample is an approximately 3.2 mm in diameter disk punched from a blood sample dried on an adsorbent matrix such as a cellulose collection paper. In addition to the sample handling being very simple, there are further advantages to the use of dried blood samples. The stability of the blood sample poses no problem, and dried blood spots are easily stored. Furthermore any errors due to pipeting are circumvented. The results of the assays using the extracted DNA as described herein are furthermore not affected by differences in the sample quality or quantity.

In one embodiment, a dried blood sample is a sample from a newborn human. The traditional method for newborn testing starts with the collection of a small amount of blood from the newborn within the first postnatal days. The sample is adsorbed onto a piece of filter paper to provide a dried blood spot. This sample is sent to the laboratory, where a small disk, about 3 mm, is punched out from the spot for DNA extraction and analysis. The use of paper cards for the collection of blood samples is also particularly suitable for the storage and transportation of blood samples at the time of epidemiologic studies or for screening a population. Advantages of dried blood samples are the ease of collection, the low volume of storage, and the transport without need for refrigeration.

An exemplary elution method includes:
1. Add 30-60 μL of one-step elution buffer to a well of a 96-well plate containing a 3.2 mm dried blood disk.
2. Cover the plate.
3. Optionally centrifuge the plate at 3700 rpm for 5 minutes.
4. Heat the plate at 95° C. for 25 minutes.
5. Optionally, cool the eluted DNA solution at a temperature of at least about 4° C. for at least 5 minutes.
6. Optionally centrifuge the plate at 3700 rpm for 5 minutes.

PCR is an enzymatic amplification reaction used routinely to amplify one or more targeted nucleic acid sequences within a sample or mixture of nucleic acids. This process is disclosed in U.S. Pat. No. 4,965,188 to Mullis. For each target nucleic acid sequence to be amplified in this process, separated complementary strands of nucleic acid are treated with two primers selected to be substantially complementary to portions of the target nucleic acid within the two strands. A thermostable enzyme (a polymerase) is generally used to extend the primers to form complementary primer extension products. When these extension products are separated into their complementary strands, they serve as templates to extend the complementary primer into the target nucleic acid sequence. When separated, these target nucleic acid sequences in turn act as templates for synthesis of additional nucleic acid sequences. The PCR amplification process involves a series of simple steps. These include temperature cycling to cause hybridization of primers and templates, polymerase mediated synthesis of the primer extension products, and separation and subsequent annealing of the strands of template strands and the synthesized target nucleic acid sequences. Thus, there is an exponential increase in the amount of targeted nucleic acid sequences synthesized. PCR amplification is a very sensitive process. Therefore, a very high purity of starting sample is necessary.

Since the development of PCR, many variations of this enzymatic amplification have been developed, including allele-specific PCR for the detection of single nucleotide polymorphisms, asymmetric PCR in which one strand of a DNA hybrid is preferentially amplified, hot-start PCR, multiplex-PCR in which multiple primer sets are used to produce different sized amplicons in a single reaction, Tetra-primer ARMS-PCR, or a quantitative PCR reaction, for example.

In one embodiment, the analysis following DNA elution is a quantitative PCR reaction also called a real-time PCR analysis of the eluted DNA. The real-time polymerase chain reaction, also called quantitative real time polymerase chain reaction (Q-PCR/qPCR/qrt-PCR) or kinetic polymerase chain reaction (KPCR), is a laboratory technique based on the PCR, which is used to amplify and simultaneously quantify a targeted DNA molecule. It enables both detection and quantification (as absolute number of copies or relative amount when normalized to DNA input or additional normalizing genes) of one or more specific sequences in a DNA sample.

In another embodiment, a kit for the detection of CMV a blood sample comprises

a DNA elution buffer,

nucleic acid amplification reagents,

a primer pair for amplification of CMV, comprising SEQ ID NO: 1 and SEQ ID NO:2, and

instructions for use.

The kit optionally further comprises the probe of SEQ ID NO: 3. In addition, the kit optionally further comprises the primers of SEQ NOs. 4 and 5 and the probe of SEQ ID NO:6 for the detection of SCID; and the β-actin control primers and probe of SEQ ID NOs: 7, 8 and 9.

In one embodiment, the DNA elution buffer is the one-step DNA elution buffer described herein.

As used herein, the term “nucleic acid amplification reagents” refers to conventional reagents that are employed in amplification procedures (such as, but not limited to, polymerase chain reaction (PCR), reverse transcription PCR (RT PCR), etc.) which are well known and may include, but are not limited to, one or more enzymes that have polymerase activity or reverse transcriptase activity, enzyme cofactors (such as magnesium or manganese; salts; nicotinamide adenine dinucleotide (NAD)), salts, buffers and deoxynucleotide triphosphates (dNTPs, for example, deoxyadenine triphosphate, deoxyguanine triphosphate, deoxycytosine triphosphate and deoxythymine triphosphate) and other reagents that modulate the activity of the enzymes or the specificity or sensitivity of the primers or probes.

Instructions include the time and temperature for DNA elution, and a description of the primers and markers in the kit. In one embodiment, the kit includes markers and primers suitable for the detection of SCID, as described above.

In one embodiment, the enzymatic amplification reaction contains primers for the amplification of a marker for a genetic disorder in addition to CMV, such as a genetic disorder tested in newborn screening. Genetic disorders tested using DNA samples in new born screening include SCID, cystic fibrosis, Sickle Cell disease, and galactosemia. In one embodiment, the genetic disorder is SCID and the primers are SEQ ID NO: 4 and SEQ ID NO: 5. When the PCR reaction is a quantitative PCR reaction, the reaction also includes the probe of SEQ ID NO: 6. A β-actin primer pair and probe serves as an optional control for DNA elution from the dried blood spots.

The invention is further illustrated by the following non-limiting examples.

### Example

### Detection of CMV DNA Using a One-Step DNA Isolation Buffer and Real-Time PCR Detection

DNA extraction was done in a 96-well plate format. A 3.2 mm disk of DBS was punched into a MicroAmp™ Optical 96-Well Reaction Plate well (Applied Biosystems, Foster City, Calif.) using a standard MultiPuncher (PerkinElmer, Waltham, Mass.). 54 μL of the DNA Elution Solution was then added to each well, and the plate heated at 99° C. for 25 minutes followed by incubation at 4° C. for 10 minutes. The DNA Elution Solution was: 15 mM Tris-Base, 10 mM KCl, and 5 mM KOH, pH 11.5.

RT-qPCR for detecting CMV DNA was performed in a total volume of 20 μl, containing 1× TaqMan® ENVIRONMENTAL MASTER MIX 2.0 (Applied Biosystems, Foster City, Calif.), 0.5 μM of CMV primers, and 0.15 μM TaqMan® probes, 0.8 μL of 1% bovine serum albumin (BSA; New England Biolabs, Ipswich, Mass.). 6 μL of DNA extract was used for the assay.

The reactions were carried out on an ABI 7900HT Fast Real-Time PCR System (Applied Biosystems, Foster City, Calif.) and underwent 1 cycle of 2 minutes at 50° C., 1 cycle of 10 minutes at 95° C., 45 cycles of 30 seconds at 95° C. and 40 seconds at 60° C.

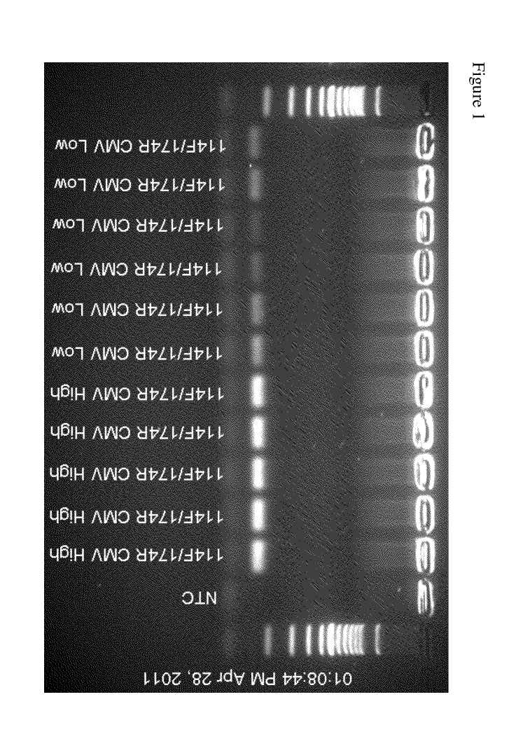

In a first experiment, samples were spiked with CMV to determine the detection limit of the method disclosed herein. In the experiment shown in FIG. 1, PCR was done on DNA extracts from CMV spiked dried filter paper. In the figure, CMV Low is estimated at 2-6 copies per sample and CMV High is estimated at 38-119 copies per sample. The assay is able to detect CMV DNA as low as 5 copies (sensitivity), and distinguish low CMV DNA copies from high CMV DNA copies (quantitative).

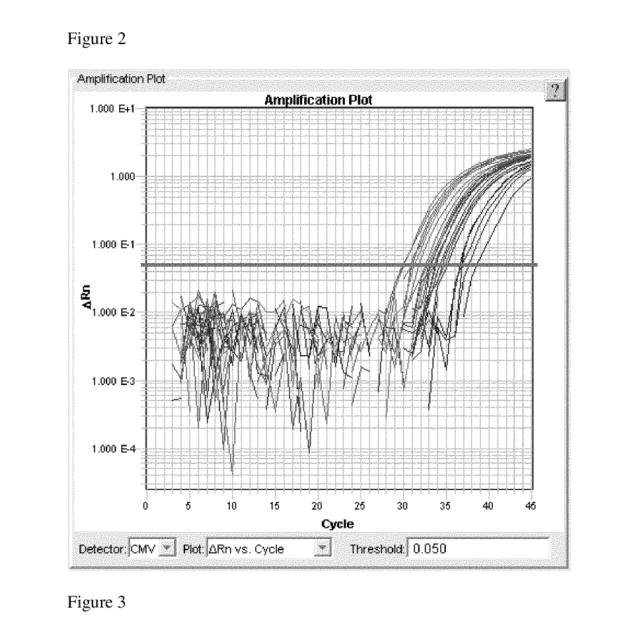

FIG. 2 shows the results of a real-time PCR experiment done on filter paper spiked with 6.5-650 copies of CMV DNA. This shows that the assay has sufficient linearity.

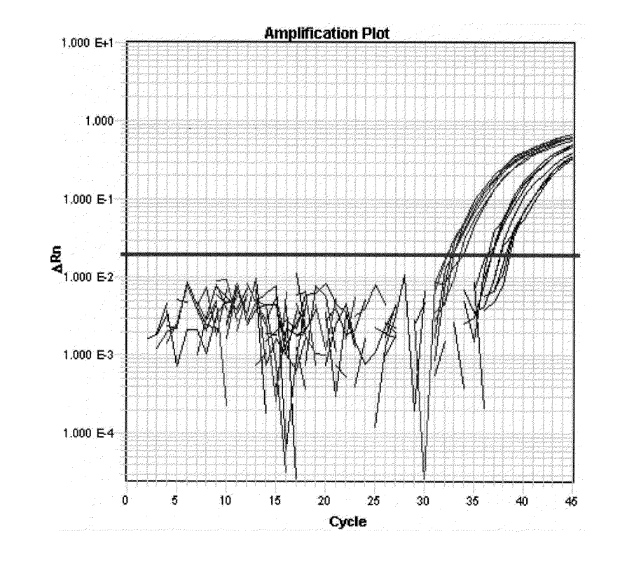

FIG. 3 shows the results of a real-time PCR experiment done on dried blood spots. It is feasible to detect CMV DNA on routine newborn screening specimens.

The following table shows the results of an assay in which CMV and SCID were determined in the same assay:

| CMV DNA | | | | TREC | | | |
|---|---|---|---|---|---|---|---|
| Copies | Singleplex | Multiplex | CV(%) | Copies | Singleplex | Multiplex | CV(%) |
| 6.5 | 38.79 | 38.24 | 1.01 | 8 | 36.05 | 36.3 | 0.49 |
| 65 | 35.18 | 35.30 | 0.24 | 80 | 32.52 | 32.67 | 0.33 |
| 130 | 34.17 | 34.19 | 0.04 | 800 | 29.39 | 29.49 | 0.24 |
| 650 | 32.38 | 32.97 | 1.28 | 8000 | 25.78 | 26.01 | 0.63 |

Thus, using the primers disclosed herein, CMV and SCID can be detected in the same sample. The assay is able to detect CMV DNA and TREC simultaneously without compromising either CMV DNA or TREC amplification efficiency.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of screening for CMV in DNA isolated from a blood sample comprising
amplifying a sequence of CMV DNA in the DNA isolated from the blood sample by contacting the DNA isolated from the blood sample with primers that are 15 to 30 nucleotides in length and comprise SEQ ID NO: 1 and SEQ ID NO: 2 under amplification conditions to produce an amplification product, and
detecting the presence of the amplification product as an indication of the presence of CMV in the blood sample.

2. The method of claim 1, wherein the CMV amplification primers comprise SEQ ID NO: 3 and SEQ ID NO: 4.

3. The method of claim 1, wherein amplifying is a real-time PCR amplification and the DNA is also contacted with a probe that is 15 to 30 nucleotides in length and comprises SEQ ID NO: 5 under conditions to produce an amplification product, wherein the probe optionally comprises a detectable label.

4. The method of claim 3, wherein the probe comprises SEQ ID NO: 6.

5. The method of claim 1, wherein the DNA isolated from the blood sample is prepared by a method that does not include washing.

6. The method of claim 1, wherein the DNA isolated from the blood sample is produced by a method comprising
adding a one-step elution buffer having a pH of about 9 to about 13 to the blood sample to form a mixture,
heating the mixture at 90° C. to 99° C. for a time sufficient to elute the DNA from the blood sample to form an eluted DNA solution, and
optionally cooling the eluted DNA solution at a temperature of at least about 4° C. for at least 5 minutes.

7. The method of claim 6, wherein the one-step elution buffer consists of 5 to 22.5 mM potassium in the form of KOH, KCl, or a combination thereof, and 7.5 to 30 mM of a base having a buffering range of 7.0 to 9.5, wherein the pH of the one-step elution buffer is about 9 to about 13.

8. The method of claim 7, wherein the base having a buffering range of 7.0 to 9.5 is selected from the group consisting of Tris base, DIPSO (3-[N,N-bis(2-Hydroxyethyl)amino]-2-hydroxypropanesulfonic Acid Sodium Salt), MOBS (3-(N-morpholino)propanesulfonic acid), TAPSO (3-[N-Tris(hydroxymethyl)methylamino]-2-hydroxypropanesulfonic Acid), HEPPSO (4-(2-Hydroxyethyl)piperazine-1-(2-hydroxypropanesulfonic acid), POPSO (piperazine-1,4-bis(2-hydroxypropanesulfonic acid)dihydrate), TEA (triethanolamine), EPPS (4-(2-hydroxyethyl)piperazine-1-propanesulfonic acid), TRICINE (N-[Tris(hydroxymethyl)methyl]glycine, 3-[(3-Cholamidopropyl)dimethylammonio]propanesulfonic acid), Glycylglycine, BICINE (N,N-Bis(2-hydroxyethyl)glycine), HEPBS (N-(2-Hydroxyethyl)piperazine-N′(4-butanesulfonic acid), TAPS(N-Tris(hydroxymethyl)methyl-3-aminopropanesulfonic Acid), AMPD (2-Amino-2-methyl-1,3-propandiol), TABS (N-tris[hydroxymethyl]methyl-4-aminobutanesulfonic acid), AMPSO (N-(1,1-dimethyl-2-hydroxyethyl)-3-amino-2-hydroxy-propanesulfonic acid), CHES (N-Cyclohexyl-2-aminoethanesulfonic acid), and combinations thereof.

9. The method of claim 6, wherein the one-step elution buffer consists of 2.5 to 10 mM KOH, 7.5 to 30 mM Tris base and 2.5 to 12.5 mM KCl, and wherein the pH is 11 to 12.

10. The method of claim 6, wherein the mixture is heated at 94° C. to 99° C.

11. The method of claim 1, wherein the blood sample is a dried blood sample on an adsorbent matrix.

12. The method of claim 11, wherein the blood sample is from a newborn human.

13. The method of claim 6, wherein the blood sample is a dried blood sample on an adsorbent matrix.

14. The method of claim 1, further comprising
simultaneously amplifying a SCID sequence in the DNA isolated from the blood sample by contacting the DNA isolated from the blood sample with primers that are 15 to 30 nucleotides in length and comprise SEQ ID NO: 7 and SEQ ID NO: 8 under amplification conditions to produce an SCID amplification product, and
detecting the presence of the SCID amplification product as an indication of the presence of SCID in the blood sample.

15. The method of claim 14, wherein amplifying is a real-time PCR amplification and the DNA is also contacted with a probe of 15 to 30 nucleotides in length and comprising SEQ ID NO: 11 under conditions to produce an amplification product, wherein the probe optionally comprises a detectable label.

16. A kit for the detection of CMV a blood sample comprising
a DNA elution buffer,
nucleic acid amplification reagents,
a primer pair for amplification of CMV, wherein the primer pair are 15 to 30 nucleotides in length and comprise SEQ ID NO: 1 and SEQ ID NO: 2, and
instructions for use.

17. The kit of claim 16, wherein the primer pair comprises SEQ ID NO: 3 and SEQ ID NO: 4.

18. The kit of claim 16, further comprising a probe that is 15 to 30 nucleotides in length and comprises SEQ ID NO: 5.

19. The kit of claim 16, further comprising primers that are 15 to 30 nucleotides in length and comprise SEQ ID NO: 7 and SEQ ID NO: 8 for the detection of SCID.

20. The kit of claim 16, wherein the DNA elution buffer is a one-step elution buffer having a pH of about 9 to about 13.

21. The kit of claim 20, wherein the one-step elution buffer consists of 5 to 22.5 mM potassium in the form of KOH, KCl, or a combination thereof, and 7.5 to 30 mM of a base having a buffering range of 7.0 to 9.5, wherein the pH of the one-step elution buffer is about 9 to about 13.

22. The kit of claim 20, wherein the one-step elution buffer consists of 2.5 to 10 mM KOH, 7.5 to 30 mM Tris base and 2.5 to 12.5 mM KCl, and wherein the pH is 11 to 12.


SEQUENCE LISTING (15 sequences)

SEQ ID NO: 1 - DNA, 13 bp, Artificial Sequence
  primer
gccggcggtatcg

SEQ ID NO: 2 - DNA, 14 bp, Artificial Sequence
  primer
gagcccgactttac

SEQ ID NO: 3 - DNA, 17 bp, Artificial Sequence
  primer
cagccggcggtatcgat

SEQ ID NO: 4 - DNA, 18 bp, Artificial Sequence
  primer
ccgagcccgactttacca

SEQ ID NO: 5 - DNA, 14 bp, Artificial Sequence
  probe
cttgttgcggtact

SEQ ID NO: 6 - DNA, 18 bp, Artificial Sequence
  probe
atcttgttgcggtactgg

SEQ ID NO: 7 - DNA, 20 bp, Artificial Sequence
  primer
acacctctggtttttgtaaa

SEQ ID NO: 8 - DNA, 15 bp, Artificial Sequence
  primer
caggtgcctatgcat

SEQ ID NO: 9 - DNA, 24 bp, Artificial Sequence
  primer
tgacacctctggtttttgtaaagg

SEQ ID NO: 10 - DNA, 19 bp, Artificial Sequence
  primer
tgcaggtgcctatgcatca

SEQ ID NO: 11 - DNA, 12 bp, Artificial Sequence
  probe
cactcctgtgca

SEQ ID NO: 12 - DNA, 16 bp, Artificial Sequence
  probe
cccactcctgtgcacg

SEQ ID NO: 13 - DNA, 20 bp, Artificial Sequence
  primer
atttccctctcaggaatgga

SEQ ID NO: 14 - DNA, 23 bp, Artificial Sequence
  primer
cgtcacacttcatgatggagttg

SEQ ID NO: 15 - DNA, 18 bp, Artificial Sequence
  probe
gtggcatccacgaaacta